(19) United States
(12) Patent Application Publication
(10) Pub. No.: US 2018/0369113 A1
(43) Pub. Date: Dec. 27, 2018

(54) PERSONAL CARE FORMULATIONS COMPRISING CARMIN PROTEIN

(71) Applicant: BOTANECO INC., Calgary (CA)

(72) Inventors: Shuanghui Liu, Calgary (CA); Kent Shafer, Calgary (CA); Stewart John Campbell, Cochrane (CA); Yves Lanctot, Deux-Montagnes (CA); Geoffrey Brooks, Reno, NV (US)

(73) Assignee: BOTANECO INC., Calgary (CA)

(21) Appl. No.: 15/781,695

(22) PCT Filed: Dec. 8, 2016
(86) PCT No.: PCT/CA2016/051439

§ 371 Date: Jun. 5, 2018

Related U.S. Application Data
(60) Provisional application No. 62/265,128, filed on Dec. 9, 2015.
(60) Provisional application No. 62/324,953, filed on Apr. 20, 2016.

(51) Int. Cl. A61K 8/64; A61Q 5/00; A61Q 5/02; A61Q 5/12; A61Q 19/10

(52) U.S. Cl. CPC A61K 8/64; A61Q 5/002; A61Q 19/10; A61Q 5/12; A61Q 5/02

(57) ABSTRACT

Personal care products comprising a carmin protein are provided. The carmin protein may be obtained in a plant extract obtainable from plants belonging to the genus Carthamus. The personal care products may be used to prevent or repair damaged hair.

### RELATED APPLICATIONS

This Application is a National Stage Application of PCT/CA2016/051439, and claims priority benefit of U.S. Provisional Application No. 62/265,128, filed on Dec. 9, 2015 and U.S. Provisional Patent Application No. 62/324,953, filed on Apr. 20, 2016, both of which are incorporated fully herein by reference in their entirety for all purposes.

### FIELD OF THE DISCLOSURE

The present disclosure relates to personal care formulations, and notably to personal care formulations comprising proteins from plant extracts.

### BACKGROUND OF THE DISCLOSURE

The following paragraphs are provided by way of background to the present disclosure. They are not however an admission that anything discussed therein is prior art or part of the knowledge of persons skilled in the art.

The personal care industry in recent years has significantly reduced the use of synthetic ingredients in favor of natural ingredients to constitute personal care products. These natural ingredients are deemed desirable by consumers, as they are frequently less damaging to the body, as well as to the environment. In many instances, suitable replacements for ingredients that have historically been of fossil fuel origin have been found. Examples include the replacement of mineral oils, silicones, and petrochemically-derived synthetic esters with vegetable oils and natural esters, synthetic fragrances with essential oils, and petrochemical preservatives with certain natural extracts.

One particular challenge facing formulators of natural personal care products is the identification of natural ingredients exhibiting properties resulting in skin tissue or hair improvement. Thus, there are, for example, few natural ingredients that have performance and use characteristics appropriate for preventing or repairing hair damage, incurred, for example, as a result of exposure to chemical agents used in hair care products, such as hair dyes and bleaching products. Accordingly, there is a need in the art to identify and develop natural ingredients that have performance and use characteristics suitable for the formulation of personal care products, including hair care products, and other products beneficial to skin tissue.

### SUMMARY OF THE DISCLOSURE

The following paragraphs are intended to introduce the reader to the more detailed description that follows and not to define or limit the claimed subject matter of the present disclosure.

In one aspect, the present disclosure relates to personal care products formulated using proteins derived from plants or plant extracts.

In another aspect, the present disclosure relates to proteins and protein extracts obtainable or obtained from plants belonging to the genus Carthamus.

In another aspect, the present disclosure relates to the use of proteins and protein extracts as ingredients in the formulation of personal care products.

Accordingly, the present disclosure provides, in at least one embodiment, a personal care formulation comprising a carmin protein and a diluent, carrier or excipient suitable for use in a personal care formulation.

In one embodiment, the carmin protein is in a protein extract obtainable or obtained from a plant belonging to the genus Carthamus.

In one embodiment of the present disclosure, the plant belonging to the genus Carthamus is Carthamus tinctorius.

In one embodiment of the present disclosure, the personal care formulation is a hair care formulation.

In one embodiment, the hair care formulation is a formulation for preventing or repairing damaged hair.

In one embodiment, the hair care formulation is a formulation for preventing or repairing damaged hair, as a result of treatment with another hair care product.

In one embodiment, of the present disclosure, the personal care formulation is a skin care formulation.

In another aspect, the present disclosure provides, in at least one embodiment, a method of formulating a personal care formulation comprising:
(a) providing a carmin protein;
(b) providing a diluent, carrier or excipient; and
(c) contacting the carmin protein with the diluent, carrier or excipient to formulate a personal care formulation.

In yet another aspect, the present disclosure provides, in at least one embodiment, a use of a carmin protein as an ingredient to prepare a personal care formulation.

The present disclosure further provides, in at least one embodiment, a use of a carmin protein to prepare a hair care formulation.

The present disclosure further provides, in at least one embodiment, a use of a formulation comprising carmin protein for personal care.

The present disclosure further provides, in at least one embodiment, a use of a formulation comprising carmin protein for hair care.

Other features and advantages or the present disclosure will become apparent from the following detailed description. It should be understood, however, that the detailed description, while indicating preferred implementations of the present disclosure, are given by way of illustration only, since various changes and modification within the spirit and scope of the disclosure will become apparent to those of skill in the art from the detailed description.

### DETAILED DESCRIPTION OF THE DISCLOSURE

Various compositions and methods will be described below to provide an example of an embodiment of each claimed subject matter. No embodiment described below limits any claimed subject matter and any claimed subject matter may cover methods, processes, or compositions that differ from those described below. The claimed subject matter is not limited to compositions or methods having all of the features of any one composition, method, or process described below or to features common to multiple or all of the compositions, or methods described below. It is possible that a composition, method or process described below is not an embodiment of any claimed subject matter. Any subject matter disclosed in a composition, method or process described below that is not claimed in this document may be the subject matter of another protective instrument, for example, a continuing patent application, and the applicants, inventors or owners do not intend to abandon, disclaim or dedicate to the public any such subject matter by its disclosure in this document.

All publications, patents, and patent application are herein incorporated by reference in their entirety to the same extent as if each individual publication, patent or patent application was specifically and individually incorporated by reference in its entirety.

### Definitions:

“Carmin”, and “carmin protein”, as may be interchangeably used herein, refers to the oligomultimeric protein compound composed of its constituent polypeptides obtainable or obtained from the seeds of plants belonging to the genus Carthamus, substantially representing the 12S total protein sedimentation fraction, having a molecular weight of approximately 300 kDa, and corresponding with the protein further described by Latha. T. S. and Prakash, V. in: Die Nahrung 30, 833-844 (1986).

“Carmin protein extract”, as used herein, refers to a protein fraction obtainable or obtained from the seed of Carthamus and comprising carmin.

“Carthamus”, as used herein, refers to a plant belonging to the biological genus Carthamus and includes, without limitation, the species Carthamus tinctorius, also known as safflower, Carthamus alexandrinus, Carthamus arborescens, Carthamus baeticus, Carthamus creticus, Carthamus flavescens, Carthamus glaucus, Carthamus lanatus, Carthamus leucocautos, Carthamus oxyacantha, Carthamus palaestinus and Carthamus turkestanicus, and further includes all plant cultivars and varieties belonging to the aforementioned.

“Hair care product” and “hair care formulation”, as may be used interchangeably herein, refer to a personal care product suitable for cleaning, cleansing, maintaining, protecting, modifying the appearance, beautifying, remediating or repairing mammalian hair, including human hair.

“Personal care product” and “personal care formulation”, as may be used interchangeably herein, refer to a composition suitable for cleaning, cleansing, maintaining, protecting, repairing, remediating, modifying the appearance or beautifying the exterior surface area of the human body, including without limitation, the skin, hair, teeth, nails, cuticles and lips, by topical application of the product to the surface area of the human body. Where reference is made to hair and hare care products, the term can further include mammalian hair.

“Substantially pure,” as interchangeably used herein, in relation to a preparation or extract refers to a preparation or extract wherein at least about from about 45% (w/w) to about 95% (w/w) of the preparation exhibits a single type of protein, or from about 60% (w/w) to about 95% (w/w), or about 70% (w/w) to about 95% (w/w), or about 80% (w/w) to about 95% (w/w), or at least about 95%. A substantially pure protein will typically comprise about 45%, 50%, 60%, 70%, 80% or 90% (w/w) of a protein preparation, more usually about 95% (w/w), and preferably will be over 99% (w/w) pure. Protein purity or homogeneity may be indicated by a number of means well known in the art, such as proximate analysis, polyacrylamide gel electrophoresis of a protein sample, followed by visualizing a single polypeptide band upon staining the gel with a stain well known in the art. For certain purposes, higher resolution may be provided by using HPLC or other means well known in the art for purification and quantification of proteins.

“Stable carmin protein”, as used herein, refers to a carmin protein preparation or extract that has been prepared so that the carmin protein preparation or extract does not undergo any reactions adversely affecting the use or performance characteristics of the protein preparation extract when it is stored for longer periods of time.

By “formulating carmin protein to form a personal care product” it is meant that the carmin protein is contacted with at least one other compound, notably a diluent, excipient or carrier, and mixed, homogenized or prepared until a personal care product is formed.

It should be noted that terms of degree such as “substantially”, “essentially” “about” and “approximately” as used herein mean a reasonable amount of deviation of the modified term such that the end result is not significantly changed. These terms of degree should be construed as including a deviation of the modified term if this deviation would not negate the meaning of the term it modifies.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

### General Implementation:

As hereinbefore mentioned, the present disclosure provides personal care formulations comprising proteins, including in natural extracts obtainable or obtained from plants belonging to the genus Carthamus, and methods of making and using such formulations. The personal care formulations of the present disclosure comprise a carmin protein and may be prepared using primarily or exclusively natural ingredients. The carmin protein may be obtained in plant extracts in a manner that preserves the integrity of the extract's primary constituent, the protein carmin. Surprisingly, upon topical application of formulations comprising carmin protein, damage to the exterior surface area of the human body, notably hair, may be prevented or repaired. Thus, for, example, shampoos and conditioners comprising carmin protein can prevent hair damage when hair is subsequently treated with chemical agents, for example, a hair bleaching agent. Formulations comprising the carmin protein preparation of the present disclosure can also provide skin moisturization benefits. Furthermore the carmin protein preparations of the present disclosure are non-irritating and hypo-allergenic, allowing use of carmin protein in a wide range of personal care products

Accordingly, the present disclosure provides, in at least one embodiment, a personal care formulation comprising a carmin protein and a diluent, carrier or excipient suitable for use in preparing a personal care formulation.

In one embodiment, the carmin protein is obtainable or obtained from a plant belonging to the genus Carthamus in a carmin protein containing protein extract.

In some embodiments, Carthamus seeds may be obtained by collecting or harvesting seed from a wild Carthamus plant or an organically or non-organically cultivated Carthamus plant or a Carthamus plant species or plant variety, including, without limitation, Carthamus tinctorius, Carthamus alexandrinus, Carthamus arborescens, Carthamus baeticus, Carthamus creticus, Carthamus flavescens, Carthamus glaucus, Carthamus lanatus, Carthamus leucocautos, Carthamus oxyacantha, Carthamus palaestinus or Carthamus turkestanicus. Carthamus varieties and cultivars may vary with respect to seed oil composition. Thus, for example, certain varieties of Carthamus tinctorius containing high oleic acid, or high linoleic acid or high stearic acid may be used. The seed may vary with respect to its stage of development and may be more or less mature and seeds from various species or varieties may be mixed. In some embodiments, the seed will be dried to obtain a dried seed containing about 10%, 5%, 2%, or 1% (w/w), exogenous water or less, and the seed thereafter is optionally cleaned and/or stored until ready to be used.

A wide variety of techniques and methodologies may be used to obtain carmin protein from Carthamus seeds, including, for example, the techniques for carmin protein isolation described in Latha and Prakash, (1984) J. Agric. Food Chem. 32:1412-16; Rao and Prakash (1989), J. Protein Chem. 8 (4): 529-48; Betchart et al. (1979) J. Food Science, 44, 1022-35; Betchart and Saunders (1978), J. Food Science, 43, 964-68; Zirwer et al., (1989), die Nahrung (10) 929-33; and U.S. Pat. No. 4,072,669.

In some embodiments, plant seeds are preconditioned by pre-heating and flaking the seeds, and thereafter homogenizing the seed, for example by comminuting, grinding or milling the seed, to obtain a homogenized plant seed material. The oil fraction, or a substantial portion thereof, may be separated from the protein fraction, for example by hydraulic pressing, or expelling the oil from the homogenized seed, and/or extracting the oil from the homogenized seed material to obtain a seed meal containing the seed protein fraction. In one embodiment, the total protein fraction is obtained from the seed meal by extraction of the seed meal using an aqueous extractant, for example, a 0.01 M phosphate buffer containing 1M NaCl at neutral pH. In one embodiment, thereafter, a fraction comprising the carmin protein is obtained by selective precipitation using, for example, 10.4% ammonium sulfate, optionally followed by lyophilization, redissolving in an aqueous buffer, for example, 50 mM phosphate buffer, pH 8.0 and additional purification techniques such as gel filtration, for example a Sepharose CL-4B column, to obtain a plant extract containing carmin protein.

In a further preferred embodiment, seed homogenization is carried out in an aqueous homogenization medium e.g. water or an aqueous buffer, and thereafter the insoluble plant seed material, including, for example, insoluble carbohydrate material, and insoluble seed proteins, is removed through a solid-liquid separation process step, for example a gravity based methodology, using e.g. decanter. Within the liquid fraction, the oil fraction is then separated from the aqueous protein fraction, for example, by gravity centrifugation. The aqueous protein fraction thus obtained is a plant seed extract containing substantial amounts of carmin protein. The protein fraction may optionally be further enriched for carmin protein using protein purification techniques including membrane filtration, gel filtration and other techniques known to those in the art.

In this manner, a carmin protein extract from which a substantial amount of seed constituents has been removed may be obtained. Preferably, a carmin protein extract is obtained from which a substantial amount of endogenous seed oil has been removed. Preferably, the carmin protein extract is an extract from which at least about 50% (w/w) of the total endogenous plant seed oil has been removed. More preferably at least about 60%, 70%, 80%, 85%, 90%, or at least about 95% (w/w), of the total endogenous seed oil has been removed from the protein extract. In some embodiments, the carmin protein extract is an extract comprising less than about 5% (w/w) of total endogenous plant seed oil. In other embodiments, the carmin protein extract comprises less than about 10%, 15%, or 20% (w/w), of the endogenous plant seed oil.

In some embodiments, a carmin protein extract comprising some endogenous seed oil may be obtained, for example a carmin protein extract comprising about 1%, 2%, 3%, 4% 5%, 6%, 7%, 8%, 9%, 10%, 15%, or 20% (w/w) of endogenous seed oil.

In some embodiments, by modulating of the concentration of endogenous seed oils, certain properties of the carmin protein extract may be modulated. Thus, in certain embodiments, by modulating the amount of oleic acid in the carmin preparation, for example, by selection of a high omega-9 fatty acid Carthamus cultivar, for example, a high oleic acid Carthamus tinctorius cultivar, as a carmin protein source, the viscosity of the preparation may be modulated. Furthermore, high oleic acid Carthamus cultivars may be selected to provide for oxidative stability.

In some embodiments, a substantial amount of the seed carbohydrate has been removed. In some embodiments, the carmin protein extract is a protein extract containing less than about 15%, 12.5%, 10%, 9%, 8% or 7% (w/w) carbohydrate.

In some embodiments, the carmin protein extract is substantially pure, and comprises from about 45% to about 95% (w/w) carmin protein, and no more than from about 5% to about 55% (w/w) other seed proteins, or from about 70% to about 95% (w/w) carmin protein, and no more than from about 5% to about 30% (w/w) of other seed protein, or no more than from about 80% to about 95% (w/w) carmin protein, and no more than from about 5% to about 20% (w/w) of other seed protein, or no more than about 5% (w/w) other seed protein.

In some embodiments, the carmin protein extract is a substantially pure protein fraction and comprises at least about 45%, 50%, 60%, 70%, 80%, 90%, 95%, or at least about 99% (w/w) carmin protein.

In some embodiments, the carmin protein extract is a protein extract comprising the following approximate amino acid composition:

| | Amino acid composition |
|---|---|
| Amino Acid | (range as a % of total protein) |
| Aspartic Acid | 9-11 |
| Glutamic Acid | 20-24 |
| Serine | 4-5 |
| Glycine | 5-6 |
| Histidine | 0.5-6 |
| Arginine | 11-12 |
| Threonine | 2-4 |
| Alanine | 4-5 |
| Proline | 3-5 |
| Tyrosine | 3-4 |
| Valine | 4-5 |
| Methionine | 1-3 |
| Cystine | 1-2 |
| Isoleucine | 3-5 |
| Leucine | 6-8 |
| Phenylalanine | 4-6 |
| Lysine | 2-4 |
| Tryptophan | 1-2 |

It should be noted that the carmin protein extracts may be analyzed using a variety of chemical and analytical assays and techniques, including, for example, by proximate analysis, amino acid composition analysis, sodium dodecyl sulfate (SDS) polyacrylamide gel electrophoresis or non-denaturing polyacrylamide gel electrophoresis, mass spectrometry (MS) techniques, nuclear magnetic resonance (NMR) techniques, and functional property analysis.

Further guidance with respect to how plant extracts comprising carmin protein may be prepared is provided for example by Latha and Prakash, (1984) J. Agric. Food Chem. 32:1412-16; Rao and Prakash (1989), J. Protein Chem. 8 (4): 529-48; Betchart et al. (1979) J. Food Science, 44, 1022-35; Betchart and Saunders (1978), J. Food Science, 43, 964-68; Zirwer et al., (1989), die Nahrung (10) 929-33; and U.S. Pat. No. 4,072,669. It is noted, however, that there are many ways in which a suitable carmin protein preparation may be obtained. Those of skill in the art will readily appreciate that a wide variety of protein fractionation and isolation techniques and schemes are available and may be applied to obtain carmin protein and carmin protein extracts. The manner in which a carmin protein preparation suitable in accordance with the present disclosure is obtained is without particular restrictions and may be as desired, and the present disclosure is not limited by the process used to obtain a suitable carmin protein containing preparation.

In accordance with some embodiments, the carmin protein may be prepared by dissolving and/or diluting the carmin protein in an appropriate solvent or diluent, preferably a solvent or diluent suitable for use in the preparation of personal care formulations. Suitable solvents or diluents include polyhydric alcohols, such as glycerine, propylene glycol or propanediol. Furthermore, the pH of the carmin preparation may be adjusted using an acid or a base. Thus, in one embodiment, the pH of the carmin preparation may be adjusted to a pH of approximately 3 to 4. At such pH, the carmin protein will generally carry an overall positive charge.

In some embodiments, the carmin protein extract is prepared in the form of a powder, for example by drying an aqueous protein extract. Moisture levels in the powder may vary, however, moisture levels are typically kept below about 10%, 9%, 8%, 7%, 6%, 5%, 4%, 3%, 2% or 1% (v/w).

In accordance with some embodiments of the present disclosure, a stable carmin protein preparation or extract suitable for use as an ingredient to prepare personal care formulations is prepared. Undesirable alterations to the carmin protein preparation or extract include, without limitation, alterations caused by exposure to air, typically resulting in oxidative reactions, visible light or other forms of radiation, exposure to microorganisms, such as bacteria, fungi, mycoplasmas, viruses and the like, and further include physical separation of preparation or extract constituents resulting in a non-homogenous carmin protein preparation or extract. Diagnostic assays to evaluate the stability of carmin protein preparations or extracts include, without limitation assays capable of assessing alterations in color, odor, texture, viscosity, pH, oxidative stability, including oxidative stability of retained endogenous seed lipids, and microbial load of a carmin protein preparation or extract. Such diagnostic assays will be generally known to those of skill in the art.

In some embodiments, small amounts of stabilizing chemical agents are mixed with the carmin protein preparation or extract. These chemical agents, upon mixing, typically contain less than about 10% (w/w) of the preparation or extract, more preferably less than about 5%, about 2.5%, about 2%, or about 1% (w/w) of a stabilized carmin protein preparation or extract. The stabilizing chemical agents are generally selected to be suitable for inclusion in a personal care formulation.

In some embodiments, stabilizing chemical agents that may be used to prepare a stable carmin protein preparation or extract are anti-oxidants, acids, bases, salts or viscosity modifying agents.

In some embodiments, stabilizing chemical agents used to prepare a stable carmin protein preparation or extract are anti-oxidants, including tocopherol or rosemary extract.

In some embodiments, the carmin protein preparation or extract may be heat-treated or treated with radiation, for example ultra-violet radiation, in order to attain a stable carmin protein preparation or extract.

In some embodiments, the carmin protein preparation or extract is sufficiently stable to permit refrigerated storage (about 0° C. to 4° C.) for a period of at least 1 month, at least 2 months, at least 3 months, at least 6 months, at least 12 months, or at least 24 months. Preferably, the carmin protein preparation or extract is stable at room temperature (about 20° C. to 24° C.) for a period of at least 1 month, at least 1 month, at least 2 months, at least 3 months, at least 6 months, at least 12 months, or at least 24 months. Furthermore, preferably, a stable carmin protein preparation or extract is prepared in a manner that yields a preparation that can withstand temperature fluctuations which may occur in non-temperature controlled environments, for example such as may occur during warehouse storage or transport.

In accordance with the present disclosure, the carmin protein preparation or extract is formulated to form a personal care product. Accordingly, the present disclosure further provides, in at least one embodiment, a method of formulating a personal care formulation comprising:
(a) providing a carmin protein;
(b) providing a diluent, carrier or excipient; and
(c) contacting the carmin protein with the diluent, carrier or excipient to formulate a personal care formulation.

In some embodiments, a carmin protein, or a stabilized carmin protein, as the case may be, and another compound suitable for use in a personal care formulation, notably a diluent, carrier or excipient are provided and the carmin protein extract is contacted with such diluent, carrier or excipient. The carmin protein extract may be provided in the form of a solution, suspension or powder or in any other desirable form. The carmin protein extract and diluent, carrier or excipient are mixed, homogenized or prepared, preferably until a homogenous mixture of the diluent, carrier or excipient and carmin protein is formed, wherein such mixture is suitable for use as a personal care product. The diluent, carrier or excipient may be any suitable diluent, carrier or excipient, and in one embodiment is a diluent carrier or excipient not endogenously present in Carthamus seed, or a mixture suitable for the preparation of a personal care product. Furthermore, the diluent, carrier or excipient may be provided in any form, including, for example, as a solution, suspension, gel, liquid, solid or crystal. The quantity of the diluent, carrier or excipient may vary and depends on the type of personal care formulation that is prepared. Typically, a plurality of compounds is provided, for example 2, 3, 4, 5, 6, 7, 8, 9, 10 or more other compounds to prepare the personal care formulation. In embodiments hereof wherein a plurality of compounds is provided, such compounds may be mixed sequentially or simultaneously.

In some embodiments, a formulation suitable for use as a personal care product comprising a mixture of compounds is pre-formed, and the carmin protein preparation or extract is separately provided and incorporated in the pre-formed personal care formulation.

In some embodiments, the carmin protein preparation or extract is incorporated during formulation. In such embodiments, the carmin protein preparation or extract may be added separately or the carmin protein preparation or extract may be incorporated together with one or more other compounds.

In some embodiments, the carmin protein is chemically modified, by reacting the carmin protein with a chemical compound capable of modifying the properties of the carmin protein. Such chemical modification may be conducted prior to formulating the carmin protein.

In some embodiments, the personal care formulation comprises a two-phase liquid system comprising an oil phase and an aqueous phase, prepared to form an emulsion, including for example an oil-in-water emulsion or a water-in-oil emulsion.

The final concentration of the carmin protein preparation or extract in the personal care product may vary. In some embodiments, the carmin protein preparation or extract comprises at least 0.01% (w/w) or about 0.01% (w/w) of the personal care product. In other embodiments the carmin protein preparation or extract comprises at least 0.1% or about 0.1% (w/w), at least 0.25% or about 0.25% (w/w) at least 0.5% or about 0.5% (w/w), at least 1% or about 1% (w/w), at least 2% or about 2% (w/w), at least 2.5% or about 2.5% (w/w), at least 5% or about 5% (w/w), at least 7.5% or about 7.5% (w/w), at least 10% or about 10% (w/w) of the personal care formulation. The concentration of carmin protein preparation or extract may be optimized or adjusted, for example by preparing a plurality of sample formulations, each including a different carmin concentration, applying each sample to, for example a hair sample or an area of skin of a test subject, and evaluating the application for example with respect to improvement in appearance. Then, a concentration of carmin protein preparation or extract may be selected that provides the most desirable effect.

In accordance herewith, the personal care formulation comprises diluent, carrier or excipient suitable for preparing a personal care formulation. In some embodiments, a mixture of two or more diluent, carrier or excipient is provided.

In some embodiments, the diluent, carrier or excipient incorporated in the personal care formulations of the present disclosure are naturally occurring compounds. In view of the fact that the protein containing carmin is a natural composition, in one embodiment, the personal care composition may be formulated using only natural diluents, carriers or excipients, thus providing a natural personal care formulation.

In some embodiments, diluent, carrier or excipient incorporated in the personal care formulations of the present disclosure are synthetic compounds.

Some optional excipients are described below in relation to personal care formulation methodologies, personal care formulations and methods of using the personal care formulations.

In some embodiments, excipients incorporated in the personal care formulations of the present disclosure are emollients, including in some embodiments, oils, waxes, lipids and other water insoluble compounds. It is noted that oils and lipids may be incorporated in the formulations of the present disclosure by using exogenous oils and lipids, or in embodiments where plant extracts containing carmin protein are used, by preparing extracts comprising plant oils. In some embodiments, emollients that may be included in the personal care formulations of the present disclosure are natural compounds, including, for example, Brassica alcohol, cetyl alcohol, shea butter, safflower oil, sunflower oil, oleyl lactate, dicaprylyl ether, beeswax, carnauba wax; and/or synthetic compounds, including, for example, dimethicone, cyclopentasiloxane, C₁₂₋₁₅ alkyl benzoate. It is noted that oils incorporated in the personal care formulations of the present disclosure, may also serve as carriers of carmin protein.

In some embodiments, excipients incorporated in the personal care formulations of the present disclosure are moisturizers. In some embodiments, the moisturizers are natural compounds, including, for example, glycerin, sodium PCA, honey, Aloe barbadensis leaf extract. In other embodiments, the moisturizers are synthetic compounds, including, for example, propylene glycol.

In some embodiments, excipients incorporated in the personal care formulations are pH-modulating agents. In some embodiments, the pH-modulating agents are natural compounds, including, for example, citric acid, lactic acid, sodium hydroxide and arginine. In other embodiments, the pH-modulating compounds are synthetic compounds, including, for example, triethanolamine.

In some embodiments, excipients incorporated in the personal care formulations of the present disclosure are viscosity-modulating agents. In some embodiments, the viscosity modulating agents are natural compounds, including, for example, xanthan gum, carrageenan gum, sclerotium gum, Brassica alcohol, cellulose and cellulose derivatives. In other embodiments, the viscosity-modulating agents are synthetic agents, including, for example, carbomer, sodium acrylate copolymer and cetyl alcohol.

In some embodiments, excipients incorporated in the personal care formulations of the present disclosure are surface active agents, including, for example, cationic surfactants, including, for example, natural cationic surfactants, such as brassicyl isoleucinate esylate; and/or synthetic cationic surfactants, such as cetrimonium chloride and behentrimonium, chloride; including further, for example, anionic surfactants, including, for example, natural anionic surfactants, such as sodium coco-sulfate; and/or synthetic anionic surfactants, such as sodium laureth sulfate; including further, for example, non-ionic surfactants, including for example, natural non-ionic surfactants, such as sorbitan olivate or sorbitan oleate; and/or synthetic non-ionic surfactants, such as polysorbate 20 and PEG-100 stearate; and including, still further, zwitterionic surfactants, including for example natural zwitterionic surfactants such as decyl glucoside and lauryl glucoside; and/or synthetic zwitterionic surfactants, such as sodium cocoamphoacetate.

In some embodiments, excipients incorporated in the personal care formulations of the present disclosure are active agents. In some embodiments, the active agents are natural active agents, including, for example, bisabolol, shea butter unsaponifiables, tocopherol, and rosemary extract. In other embodiments, the active agents are synthetic active agents, including for example, palmitoyl tetrapeptide-7 and polyquaternium-10.

In some embodiments, excipients that may be incorporated in the personal care formulation include chelating agents. In some embodiments, the chelating agents are natural chelating agents, such as sodium gluconate. In other embodiments, the chelating agents are synthetic chelating agents, such as disodium EDTA.

In some embodiments, excipients that may be incorporated in the personal care formulation include proteins, other than carmin. In some embodiments, the proteins are natural proteins, such as plant proteins. It is noted that plant proteins may be incorporated in the formulations of the present disclosure by using exogenous plant proteins, or in embodiments where plant extracts containing carmin protein are used, by preparing extracts comprising, in addition to carmin protein, other plant proteins.

In some embodiments, excipients that may be incorporated in the personal care formulation are hair conditioning agents. In some embodiments, the hair conditioning agents are natural agents, such as oat peptides, hydrolyzed wheat protein, and panthenol. In other embodiments, the hair conditioning agents are synthetic agents, such as quaternium-79, hydrolyzed keratin and PEG-3 dimethicone.

In some embodiments, excipients that may be incorporated in the personal care formulation are hair fixatives. In some embodiments, the hair fixatives are natural hair fixatives such as tapioca starch, shellac, and chitosan. In other embodiments, the hair fixatives are synthetic hair fixatives such as PVP, polyquaternium-7 and PPG-12/SDM copolymer.

The final concentrations of the diluents, excipients and/or carriers comprising the personal care formulations, the pH of the personal care formulations, the viscosity of the personal care formulations, and other chemical and physicochemical properties of the personal care formulations of the present disclosure, and the manner in which the personal care formulations are constituted may vary substantially depending on the desired use and performance characteristics of the personal care formulation. Those of skill in the art will be familiar with a variety of different methodologies and techniques, for example, heating methodologies, stirring or mixing techniques, pH adjustment techniques, viscosity adjustment methodologies, and the like, all of which may be used, adjusted and/or optimized in to prepare suitable personal care formulations.

Examples 1-28 further provide specific illustrative and non-limiting personal care formulations and methodologies of preparing and using such formulations in accordance with the present disclosure.

The carmin protein preparations in accordance with the present disclosure may be used to prepare personal care formulations intended for a wide variety of different uses, including formulations intended for a specific treatment of the exterior of the human body, including, for example, formulations that prevent or treat changes to the exterior surface area of the human body, such as skin changes as result of skin aging, such as wrinkles or skin blotches, or changes caused by external factors such as, for example, sun light, wind, cold, heat, dampness, dryness, air pollution or smoke. Some exemplary personal care formulations in relation to the use of such of such formulations are described below.

In some embodiments, the personal care formulation is a hair care formulation, including, without limitation, a shampoo, a conditioner, a re-conditioner, a mousse, a gel, a hair spray, a pomade, a hair waving agent, a hair dye, a hair coloring formulation, a hair lightening formulation or a hair bleaching formulation.

In some embodiments, the personal care formulation is a hair care formulation capable of protecting hair against damage, or preventing hair from incurring damage.

In some embodiments, the personal care formulation is a hair care formulation capable of repairing or remediating damaged hair in need thereof.

It should be noted that hair damage may be caused by numerous different factors. Thus, for example, the appearance of hair may be modified by the use of hair care products, including hair dyes, lighteners, bleaches, permanent treatments and the like. The use of these products in many instances involves the use of harsh chemicals and/or heat which may damage or weaken hair. Hair damage may also result from the use of thermal hair treatment equipment, such as blow dryers and curling irons. Even repeated brushing or combing may cause hair damage. Likewise, excessive exposure to UV radiation and environmental factors, swimming pool chemicals and particulate aerial pollutants, for example, may act as causative agents of hair damage. The foregoing may result in different types of damage to human hair and may compromise the appearance thereof, including, for example split or double ends, broken hair, discoloration, scales, dryness or porosity.

In some embodiments, the personal care formulation is a hair care formulation capable of preventing, remediating, improving or repairing hair damage as a result of hair treatment with chemical agents present in a hair care product. In other embodiments, the hair care formulation is a formulation for preventing, remediating, improving or repairing damaged hair, as a result of treatment with another hair care product. In other embodiments, the personal care formulation is a formulation for the prevention of hair damage or treatment of damaged hair as a result of hair treatment with thermal hair treatment equipment or brushing or combing hair. In other embodiments, the personal care formulation is a hair care formulation capable of preventing hair damage or remediating or repairing hair damaged by environmental factors, including UV radiation, and environmental chemicals including particulate aerial pollutants and swimming pool chemicals.

In some embodiments, the hair care formulation is a rinse-off formulation in which the carmin protein has been incorporated, including, in some embodiments, a shampoo, a conditioner, a re-conditioner, a hair dye, or a hair coloring formulation, a hair lightening formulation or a hair bleaching formulation. By the term “rinse-off” it is meant that the formulation is intended to be applied to hair and shortly thereafter, for example within about 2 to 30 minutes, hair is rinsed. In other embodiments, the hair care product is a leave-on formulation, including a mousse, a gel, a hair spray, a pomade, or a hair waving agent. By the term “leave-on” it is meant that hair is not rinsed following application of the formulation.

In some embodiments, the hair care formulation is a formulation in which the carmin protein has been incorporated, including, in some embodiments, a shampoo, or a conditioner, wherein the hair care formulation protects colored or dyed hair against wash-out, fade-out and dry-out to preserve the hair color provided for by the hair dye or coloring agent.

It should be noted that, in accordance herewith, color wash-out, fade-out and dry-out, which may be analyzed in several manners, including for example by evaluating hair fibers using a spectrophotometer, for example using a Spectra Flash 600 (data color spectrophotometer), in order to evaluate the performance characteristics of the of the personal care formulations of the present disclosure.

In some embodiments, the personal care formulation is a hair care formulation capable of preventing, repairing or remediating hair displaying split or double ends, scales, broken or damaged hair fibers, dryness, discoloration or porosity.

It should be noted that, in accordance herewith, split or double ends, broken hair, discoloration, scales, dryness or porosity, may analyzed in several manners, including for example by evaluating hair fibers using scanning electron microscopy, in order to evaluate the performance characteristics of the personal care formulations of the present disclosure.

In accordance herewith, the hair care formulations may be applied to any mammalian hair type, including any human hair type including African, Asian and Caucasian hair. Furthermore, the hair care formulations may be applied to hair of individuals of any age, type, for example curly, straight, thick, thin, any color (natural or artificial), or any condition, for example, clean, dirty, or damaged hair.

In order to deliver the hair care formulation of the present disclosure to mammalian hair, including human hair, it may be delivered in any desirable manner. In some embodiments, the hair care formulation of the present disclosure is applied for a time sufficiently long to improve the strength or overall appearance of the hair. This may involve the daily application of the hair care formulation for a period of, for example, at least 3 days, at least 5 days, at least one week, or at least 2 weeks, however improvements may be observed after a single application. In some embodiments, the hair care formulations are applied to damp hair after shampooing, for example, within 30 minutes. In some embodiments, the hair care formulations are applied to damp hair following dying, bleaching, lightening or coloring hair.

In some embodiments, the personal care formulation of the present disclosure is a skin care formulation, including without limitation a skin cream, a facial cream, a skin cleanser, a day cream, a night cream, a skin toner, a lotion, a facial mask, an anti-aging cream, an anti-wrinkle cream, a cold weather cream, a foot cream, or a hand cream.

In some embodiments, the personal care formulation, is a bath and body formulation, including a body wash, a bar soap, a bath gel, or a shower gel.

In some embodiments, the personal care formulation is a sun care formulation, including, for example, a sunscreen, an after sun lotion or a tanning lotion.

In some embodiments, the personal care formulation is a make-up formulation, including, for example, a mascara, a blush, a lipstick, eye shadow, a nail polish or a make-up remover.

In some embodiments, the personal care formulation is a shaving formulation, for example, a shaving cream or a shaving gel.

As hereinbefore mentioned, in another aspect, the present disclosure further provides, in at least one embodiment, a method of formulating a personal care formulation comprising
(a) providing a carmin protein;
(b) providing a diluent, carrier or excipient; and
(c) contacting the carmin protein with the diluent, carrier or excipient to formulate a personal care formulation.

In some embodiments, the method is a method wherein the carmin protein is obtainable from Carthamus tinctorius.

In some embodiments, the method is a method wherein the carmin protein comprises less than about 40% (w/w) of other seed protein.

In some embodiments, the method is a method wherein the formulation comprises at least about 0.01% (w/w) of the carmin protein.

In some embodiments, the method is a method wherein the formulation comprises at least about 0.1% (w/w) of the carmin protein.

In some embodiments, the method is a method wherein the formulation comprises at least about 0.5% (w/w) of the carmin protein.

In some embodiments, the method is a method wherein the formulation comprises from about 0.01% (w/w) to about 10% (w/w) of the carmin protein.

In some embodiments, the method is a method wherein the carmin protein is a plant extract comprising from about 1% to about 50% endogenous Carthamus plant oils.

In some embodiments, the method is a method wherein the diluent, carrier or excipient is selected from the group consisting of emollients, moisturizers, pH-modifying agents, viscosity modifying agents, surfactants and active agents.

In some embodiments, the method is a method wherein the formulation is selected from the group consisting of a skin care formulation, a bath and body formulation, a sun care formulation, a make-up formulation or a shaving formulation.

In some embodiments, the method is a method wherein the personal care formulation is a hair care formulation.

In some embodiments, the method is a method wherein the hair care formulation is a rinse-off formulation.

In some embodiments, the method is a method wherein the hair care formulation is a leave-on formulation.

In some embodiments, the method is a method wherein the hair care product is selected from the group consisting of a shampoo, a conditioner, a re-conditioner, a mousse, a gel, a hair spray, a pomade, a hair waving agent, a hair dye, a hair coloring formulation, a hair lightening formulation and a hair bleaching formulation.

In some embodiments, the method is a method wherein the hair care formulation is product capable of preventing or repairing damaged hair.

In some embodiments, the hair care formulation is a product capable of preventing hair damage as a result of subsequent treatment with another hair care formulation. In some embodiments, the other hair care formulation is a formulation intended for hair bleaching, for example, a hydrogen peroxide containing formulation.

In some embodiments, the method is a method wherein the damaged hair is damaged as a result of exposure to hair to chemical agents, treatment with thermal hair treatment equipment, or radiation.

In some embodiments, the method is a method wherein the damaged hair displays split ends, broken hair fibers, discoloration, scales, dryness or porosity.

The present disclosure further provides, in at least one embodiment, a use of carmin protein as an ingredient to prepare a personal care formulation.

The present disclosure further provides, in at least one embodiment, a use of a plant seed extract containing carmin protein obtainable or obtained from a plant belonging to the genus Carthamus as an ingredient to prepare a personal care formulation.

The present disclosure further provides, in at least one embodiment, a use of carmin protein as an ingredient to prepare a hair care formulation.

The present disclosure further provides, in at least one embodiment, a use of carmin protein as an ingredient to prepare a hair care formulation capable of preventing damage from subsequent treatment by another hair care formulation. In some embodiments the other hair care formulation is a formulation intended for the bleaching of hair, for example, a hydrogen peroxide containing formulation. In some embodiments, the hair care formulation capable of preventing damage is a shampoo or a conditioner.

In yet another aspect, the present disclosure provides, in at least one embodiment, a use of a plant seed extract containing carmin protein obtainable or obtained from a plant belonging to the genus Carthamus as an ingredient to prepare a hair care formulation.

The present disclosure further provides, in at least one embodiment, a use of formulation comprising a carmin protein for personal care.

The present disclosure further provides, in at least one embodiment, a use of formulation comprising a plant seed extract containing carmin protein obtainable or obtained from a plant belonging to the genus Carthamus for personal care.

The present disclosure further provides, in at least one embodiment, a use of formulation comprising a carmin protein for hair care.

The present disclosure further provides, in at least one embodiment, a use of formulation comprising a plant seed extract containing carmin protein obtainable or obtained from a plant belonging to the genus Carthamus for hair care.

The above disclosure generally describes various aspects of methods and compositions of the present disclosure. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present invention:

### EXAMPLES

### Example 1

### Solubilization of Carmin Protein Extract

Spray-dried carmin protein extract has limited solubility in water alone. In order to solubilize carmin protein extract for formulation purposes, different solvents were tested for their ability to solubilize carmin protein extract. As below, the carmin protein extract was dispersed in the solvent and then added to water to ensure that the carmin protein extract was still dispersed.

| | |
|---|---|
| Solvent | 5.0% |
| Carmin protein extract | 0.5% |
| Water | 94.5% |

The solvents tested were glycerin, propylene glycol and propanediol. The carmin protein extract was sufficiently dispersed for all of the solvents tested.

### Example 2

### Preparation of a Natural Shampoo Without Carmin Protein Extract (BC-35-16-A)

| Ingredients | % |
|---|---|
| Phase A | |
| Water | 43.2 |
| Glycerin | 5.0 |
| Keltrol ® CG-SFT (Xanthan Gum) | 0.8 |
| Genuvisco ® CG-131 (Carrageenan) | 20.0 |
| Carmin Protein Extract | 0.0 |
| Phase B | |
| Plantaren ® 2000 (Decyl Glucoside) | 20.0 |
| Plantaren ® 1200 (Lauryl Glucoside) | 10.0 |
| Phase C | |
| Preservation | 1.0 |

The ingredients from Phase A were mixed together ingredients and added to the water under strong agitation with a propeller with mixing for 30 minutes. The ingredients from Phase B were added to Phase B and mixed for 10 minutes with the propeller. Phase C was subsequently added to the Phase A and B mixture and mixed for a further 10 minutes. The pH was adjusted to between 5.5 and 6.0.

### Example 3

### Preparation of a Natural Shampoo with 0.1% Carmin Protein Extract (BC-35-16-D)

| Ingredients | % |
|---|---|
| Phase A | |
| Water | 43.1 |
| Glycerin | 5.0 |
| Keltrol ® CG-SFT (Xanthan Gum) | 0.8 |
| Genuvisco ® CG-131 (Carrageenan) | 20.0 |
| Carmin Protein Extract | 0.1 |
| Phase B | |
| Plantaren ® 2000 (Decyl Glucoside) | 20.0 |
| Plantaren ® 1200 (Lauryl Glucoside) | 10.0 |
| Phase C | |
| Preservation | 1.0 |

The ingredients from Phase A were mixed together ingredients and added to the water under strong agitation with a propeller with mixing for 30 minutes. The ingredients from Phase B were added to Phase B and mixed for 10 minutes with the propeller Phase C was subsequently added to the Phase A and B mixture and mixed for a further 10 minutes. The pH was adjusted to between 5.5 and 6.0.

### Example 4

### Preparation of a Natural Shampoo with 0.5% Carmin Protein Extract (BC-35-16-E)

| Ingredients | % |
|---|---|
| Phase A | |
| Water | 42.7 |
| Glycerin | 5.0 |
| Keltrol ® CG-SFT (Xanthan Gum) | 0.8 |
| Genuvisco ® CG-131 (Carrageenan) | 20.0 |
| Carmin Protein Extract | 0.5 |
| Phase B | |
| Plantaren ® 2000 (Decyl Glucoside) | 20.0 |
| Plantaren ® 1200 (Lauryl Glucoside) | 10.0 |
| Phase C | |
| Preservation | 1.0 |

The ingredients from Phase A were mixed together ingredients and added to the water under strong agitation with a propeller with mixing for 30 minutes. The ingredients from Phase B were added to Phase B and mixed for 10 minutes with the propeller. Phase C was subsequently added to the Phase A and B mixture and mixed for a further 10 minutes. The pH was adjusted to between 5.5 and 6.0.

### Example 5

### Preparation of a Natural Conditioner without Carmin Protein Extract (BC-35-19-A)

| Ingredients | % |
|---|---|
| Phase A | |
| Water | 81.6 |
| Arginine | 0.9 |
| Phase B | |
| Brassicyl Isoleucinate Esylate | 9.0 |
| (and) Brassica Alcohol | |
| Shea Butter | 2.0 |
| Sunflower Oil | 2.0 |
| Phase C | |
| Glycerin | 3.0 |
| Carmin Protein Extract | 0.0 |
| Phase D | |
| Preservation | 1.5 |

The ingredients of Phase A were combined with mixing with a propeller and heated to 75° C. In a separate container, the ingredients of Phase B were weighed and also heated to 75° C. while mixing with a propeller. Phase B was added to Phase A under strong agitation at 500-800 rpm for 20 minutes at 75° C. After 20 minutes the agitation was changed to a side scraper and the ingredients were allowed to cool to below 35° C. In the meantime, the Phase C ingredients were mixed together until homogenous and then added to the combination of Phase A and B and subsequently mixed for 10 minutes. The Phase D ingredients were added to the Phase A, B and C mixture and subsequently mixed for a further 10 minutes. The formulation was allowed to sit for 24 hours at room temperature and the pH was adjusted to between 3.5 and 4 with citric acid or sodium hydroxide. The final viscosity of the formulation was 4,400 cps as measured with a Brookfield RVF #4 at 20 rpm.

### Example 6

### Preparation of a Natural Conditioner with 0.1% Carmin Protein Extract (BC-35-19-B)

| Ingredients | % |
|---|---|
| Phase A | |
| Water | 81.5 |
| Arginine | 0.9 |
| Phase B | |
| Brassicyl Isoleucinate Esylate | 9.0 |
| (and) Brassica Alcohol | |
| Shea Butter | 2.0 |
| Sunflower Oil | 2.0 |
| Phase C | |
| Glycerin | 3.0 |
| Carmin Protein Extract | 0.1 |
| Phase D | |
| Preservation | 1.5 |

The ingredients of Phase A were combined with mixing with a propeller and heated to 75° C. In a separate container, the ingredients of Phase B were weighed and also heated to 75° C. while mixing with a propeller. Phase B was added to Phase A under strong agitation at 500-800 rpm for 20 minutes at 75° C. After 20 minutes the agitation was changed to a side scraper and the ingredients were allowed to cool to below 35° C. In the meantime, the Phase C ingredients were mixed together until homogenous and then added to the combination of Phase A and B and mixed for 10 minutes. The Phase D ingredients were added to the Phase A, B and C mixture and mixed for 10 minutes. The formulation was allowed to sit for 24 hours at room temperature and the pH was adjusted to between 3.5 and 4 with citric acid or sodium hydroxide. The final viscosity of the formulation was 4,400 cps as measured with a Brookfield RVF #4 at 20 rpm.

### Example 7

### Preparation of a Natural Conditioner with 0.5% Carmin Protein Extract (BC-35-19-C)

| Ingredients | % |
|---|---|
| Phase A | |
| Water | 81.1 |
| Arginine | 0.9 |
| Phase B | |
| Brassicyl Isoleucinate Esylate | 9.0 |
| (and) Brassica Alcohol | |
| Shea Butter | 2.0 |
| Sunflower Oil | 2.0 |
| Phase C | |
| Glycerin | 3.0 |
| Carmin Protein Extract | 0.5 |
| Phase D | |
| Preservation | 1.5 |

The ingredients of Phase A were combined with mixing with a propeller and heated to 75° C. In a separate container, the ingredients of Phase B were weighed and also heated to 75° C. while mixing with a propeller. Phase B was added to Phase A under strong agitation, 500-800 rpm for 20 minutes at 75° C. After 20 minutes the agitation was changed to a side scraper and the ingredients were allowed to cool to below 35° C. In the meantime, the Phase C ingredients were mixed together until homogenous and then added to the combination of Phase A and B and mixed for 10 minutes. The Phase D ingredients were added to the Phase A, B and C mixture and mixed for 10 minutes. The formulation was allowed to sit for 24 hours at room temperature and the pH was adjusted to between 3.5 and 4 with citric acid or sodium hydroxide. The final viscosity of the formulation was 4,400 cps as measured with a Brookfield RVF #4 at 20 rpm.

### Example 8

### Preparation of a Synthetic Shampoo without Carmin Protein Extract (BC-35-18-A)

| Ingredients | % |
|---|---|
| Phase A | |
| Water | 63.1 |
| Carbopol ® Ultrez-20 (Acrylates/C10-30 | 0.9 |
| Alkyl Acrylate Crosspolymer) | |
| Phase B | |
| Standapol ® ES-2 (Sodium Laureth Sulfate) | 30.0 |
| Phase C | |
| Glycerin | 5.0 |
| Carmin Protein Extract | 0.0 |
| Phase D | |
| Preservation | 1.0 |

The Carbopol® Ultrez-20 of Phase A was added into the water without agitation. The mixture was allowed to sit for 5 minutes and then mixed with a propeller. Phase B was added to Phase A ingredients and mixed for 15 minutes. The pH was adjusted to 5.5-6.0 with Triethanolamine 99%. The ingredients of Phase C were mixed together ingredients and added to mixture of A+B and subsequently mixed for 10 minutes. The ingredients of Phase D were added to mixture of A+B+C and mixed for 10 minutes.

### Example 9

### Preparation of a Synthetic Shampoo with 0.1% Carmin Protein Extract (BC-35-18-B)

| Ingredients | % |
|---|---|
| Phase A | |
| Water | 63.0 |
| Carbopol ® Ultrez-20 (Acrylates/C10-30 | 0.9 |
| Alkyl Acrylate Crosspolymer) | |
| Phase B | |
| Standapol ®ES-2 (Sodium Laureth Sulfate) | 30.0 |
| Phase C | |
| Glycerin | 5.0 |
| Carmin Protein Extract | 0.1 |
| Phase D | |
| Preservation | 1.0 |

The Carbopol® Ultrez-20 of Phase A was added into the water without agitation. The mixture was allowed to sit for 5 minutes and then mixed with a propeller. Phase B was added to Phase A ingredients and mixed for 15 minutes. The pH was adjusted to 5.5-6.0 with Triethanolamine 99%. The ingredients of Phase C were mixed together ingredients and added to mixture of A+B and subsequently mixed for 10 minutes. The ingredients of Phase D were added to mixture of A+B+C and mixed for 10 minutes.

### Example 10

### Preparation of a Synthetic Shampoo with 0.5% Carmin Protein Extract (BC-35-18-C)

| Ingredients | % |
|---|---|
| Phase A | |
| Water | 62.6 |
| Carbopol ® Ultrez-20 (Acrylates/C10-30 | 0.9 |
| Alkyl Acrylate Crosspolymer) | |
| Phase B | |
| Standapol ® ES-2 (Sodium Laureth Sulfate) | 30.0 |
| Phase C | |
| Glycerin | 5.0 |
| Carmin Protein Extract | 0.5 |
| Phase D | |
| Preservation | 1.0 |

The Carbopol® Ultrez-20 of Phase A was added to the water without agitation. The mixture was allowed to sit for 5 minutes and then mixed with a propeller. Phase B was added to the Phase A ingredients and mixed for 15 minutes. The pH was adjusted to 5.5-6.0 with Triethanolamine 99%. The ingredients of Phase C were mixed together and added to mixture of A+B and subsequently mixed for 10 minutes. The ingredients of Phase D were added to mixture of A+B+C and mixed for 10 minutes.

### Example 11

### Preparation of a Synthetic Conditioner without Carmin Protein Extract (BC-35-20-A)

| Ingredients | % |
|---|---|
| Phase A | |
| Water | 80.2 |
| Phase B | |
| Incroquat ™ Behenyl TMC 25 (Cetearyl alcohol | 4.0 |
| (and) Behentrimomium chloride) (Croda) | |
| Butyrospermum Parkii (Shea Butter) (Shea Butter | 2.0 |
| Ultra Refined, Bio Botanica) | |
| Lipocol ® C (Cetyl Alcohol) | 2.0 |
| Sunflower Oil | 2.0 |
| Phase C | |
| Carthamus tinctorius (safflower) Oleosomes plus | 5.0 |
| water (Hydresia ® SF2, Botaneco) | |
| Fragrance Fructis | 0.3 |
| Phase D | |
| Glycerin | 3.0 |
| Carmin Protein Extract | 0.0 |
| Phase E | |
| Preservation | 1.5 |

The ingredients of Phase A were heated to 75° C. In a separate container, the ingredients of Phase B were weighed and also heated to 75° C. Phase B was added to Phase A under strong agitation with a propeller at 500-800 rpm for 20 minutes at 75° C. The ingredients were allowed to cool to below 45° C. In the meantime, the Phase C ingredients were mixed at 400 rpm with a propeller for 20 minutes and then added to the combination of Phase A and B and subsequently mixed for another 10 minutes. The Phase D ingredients were mixed together and added to the Phase A, B and C mixture and subsequently mixed for 10 minutes. The formulation was allowed to sit for 24 hours at room temperature and the pH was adjusted to between 3.5 and 4 with citric acid. The final viscosity of the formulation was 10,500 cps as measured with a Brookfield RVF #6 at 20 rpm.

### Example 12

### Preparation of a Synthetic Conditioner with 0.1% Carmin Protein Extract (BC-35-20-C)

| Ingredients | % |
|---|---|
| Phase A | |
| Water | 80.1 |
| Phase B | |
| Incroquat ™ Behenyl TMC 25 (Cetearyl alcohol | 4.0 |
| (and) Behentrimomium chloride) (Croda) | |
| Butyrospermum Parkii (Shea Butter) (Shea Butter | 2.0 |
| Ultra Refined, Bio Botanica) | |
| Lipocol ® C (Cetyl Alcohol) | 2.0 |
| Sunflower Oil | 2.0 |
| Phase C | |
| Carthamus tinctorius (safflower) Oleosomes plus | 5.0 |
| water (Hydresia ® SF2, Botaneco) | |
| Fragrance Fructis | 0.3 |
| Phase D | |
| Glycerin | 3.0 |
| Carmin Protein Extract | 0.1 |
| Phase E | |
| Preservation | 1.5 |

The ingredients of Phase A were heated to 75° C. In a separate container, the ingredients of Phase B were weighed and also heated to 75° C. Phase B was added to Phase A under strong agitation with a propeller at 500-800 rpm for 20 minutes at 75° C. The ingredients were allowed to cool to below 45° C. In the meantime, the Phase C ingredients were mixed at 400 rpm with a propeller for 20 minutes and then added to the combination of Phase A and B and subsequently mixed for another 10 minutes. The Phase D ingredients were mixed together and added to the Phase A, B and C mixture and subsequently mixed for 10 minutes. The formulation was allowed to sit for 24 hours at room temperature and the pH was adjusted to between 3.5 and 4 with citric acid. The final viscosity of the formulation was 10,500 cps as measured with a Brookfield RVF #6 at 20 rpm.

### Example 13

### Preparation of a Synthetic Conditioner with 0.5% Carmin Protein Extract (BC-35-20-D)

| Ingredients | % |
|---|---|
| Phase A | |
| Water | 79.7 |
| Phase B | |
| Incroquat ™ Behenyl TMC 25 (Cetearyl alcohol | 4.0 |
| (and) Behentrimomium chloride) (Croda) | |
| Butyrospermum Parkii (Shea Butter) (Shea | 2.0 |
| Butter Ultra Refined, Bio Botanica) | |
| Lipocol ® C (Cetyl Alcohol) | 2.0 |
| Sunflower Oil | 2.0 |
| Phase C | |
| Carthamus tinctorius (safflower) Oleosomes plus | 5.0 |
| water (Hydresia ® SF2, Botaneco) | |
| Fragrance Fructis | 0.3 |
| Phase D | |
| Glycerin | 3.0 |
| Carmin Protein Extract | 0.5 |
| Phase E | |
| Preservation | 1.5 |

The ingredients of Phase A were heated to 75° C. In a separate container, the ingredients of Phase B were weighed and also heated to 75° C. Phase B was added to Phase A under strong agitation with a propeller at 500-800 rpm for 20 minutes at 75° C. The ingredients were allowed to cool to below 45° C. In the meantime, the Phase C ingredients were mixed at 400 rpm with a propeller for 20 minutes and then added to the combination of Phase A and B and subsequently mixed for another 10 minutes. The Phase D ingredients were mixed together and added to the Phase A, B and C mixture and subsequently mixed for 10 minutes. The formulation was allowed to sit for 24 hours at room temperature and the pH was adjusted to between 3.5 and 4 with citric acid. The final viscosity of the formulation was 10,500 cps as measured with a Brookfield RVF #6 at 20 rpm.

### Example 14

### Consumer Assessment of Natural Shampoo Formulation with Different Levels of Carmin Protein Extract

Study participants (4-5) were asked to use the natural shampoo formulations described in examples 2-4 and then asked to rate the products on a 1 to 5 scale based on 9 separate characteristics as per the table below:

| Characteristic | | 1 | 2 | 3 | 4 | 5 | |
|---|---|---|---|---|---|---|---|
| Product | unpleasant | | | | | | great-feeling product |
| texture/feel | | | | | | | |
| Overall hair feel | unpleasant | | | | | | hair feels great |
| Greasiness | very greasy | | | | | | no greasiness |
| Shine | dull and lack | | | | | | fantastic shine |
| | luster | | | | | | |
| Softness | not soft at all | | | | | | incredible softness |
| Body/volume | flat | | | | | | lots of body/volume |
| Hair comb- | difficult/tangled | | | | | | combs easily, no |
| through/tangling | | | | | | | tangles |
| Residue | lots of residue | | | | | | no residue |
| Static | high | | | | | | no/low |

The combined results for the natural shampoo formulations are provided below.

| | 0% carmin | 0.1% carmin | 0.5% carmin |
|---|---|---|---|
| | protein | protein | protein |
| | BC-35-16-A | BC-35-16-D | BC-35-16-E |
| Product texture/feel | 3.6 | 3.0 | 2.5 |
| Overall hair feel | 4.4 | 3.0 | 3.5 |
| Greasiness | 4.2 | 4.3 | 3.8 |
| Shine | 4.4 | 3.3 | 3.3 |
| Softness | 4.4 | 3.3 | 3.3 |
| Body/volume | 3.8 | 3.5 | 3.3 |
| Hair comb-through/tangling | 3.0 | 3.0 | 3.8 |
| Residue | 3.4 | 4.8 | 3.3 |
| Static | 3.8 | 4.0 | 4.0 |

For the natural shampoos, the subjects preferred either formulations with no carmin protein extract (BC-35-16-A—Example 2) with respect to product texture/feel, overall hair feel, shine, softness, body/volume, the 0.1% carmin protein extract formulation (BC-35-16-D—Example 3) was preferred with respect to greasiness, residue and static, the 0.5% carmin protein extract formulation (BC-35-16-E—Example 4) was ranked highest for hair comb-through/tangling.

### Example 15

### Consumer Assessment of Natural Conditioner Formulation with Different Levels of Carmin Protein Extract

4-5 study participants were asked to use the natural conditioner formulations described in examples 5-7 and then asked to rate the products on a 1 to 5 scale based on 9 separate characteristics based on the scale described in Example 14. The combined results for the natural conditioner formulations are provided below.

| | 0% carmin | 0.1% carmin | 0.5% carmin |
|---|---|---|---|
| | protein | protein | protein |
| | BC-36-19-A | BC-36-19-B | BC-36-19-C |
| Product texture/feel | 3.4 | 4.3 | 4.5 |
| Overall hair feel | 4.5 | 3.8 | 4.0 |
| Greasiness | 4.3 | 4.2 | 4.0 |
| Shine | 3.8 | 4.0 | 3.5 |
| Softness | 4.2 | 3.8 | 3.8 |
| Body/volume | 3.0 | 3.8 | 3.5 |
| Hair comb-through/tangling | 3.8 | 4.2 | 3.8 |
| Residue | 3.6 | 4.0 | 3.8 |
| Static | 3.6 | 4.0 | 3.5 |

For the natural conditioners, the subjects preferred either formulations with no carmin protein extract (BC-36-19-A—Example 5) for overall hair feel and softness, the 0.1% carmin protein extract formulation (BC-36-19-B—Example 6) was the preferred for greasiness, shine, body/volume, hair comb-through/tangling, residue and static, the 0.5% carmin protein extract formulation (BC-36-19-C—Example 7) was the preferred for product texture/feel.

### Example 16

### Consumer Assessment of Synthetic Shampoo Formulation with Different Levels of Carmin Protein Extract

4-5 study participants were asked to use the natural conditioner formulations described in examples 8-10 and then asked to rate the products on a 1 to 5 scale based on 9 separate characteristics based on the scale described in Example 14. The combined results for the synthetic shampoo formulations are provided below.

| | 0% carmin | 0.1% carmin | 0.5% carmin |
|---|---|---|---|
| | protein | protein | protein |
| | BC-35-18-A | BC-35-18-B | BC-35-18-C |
| Product texture/feel | 4.3 | 4.0 | 4.0 |
| Overall hair feel | 3.8 | 3.5 | 4.3 |
| Greasiness | 3.8 | 4.5 | 4.7 |
| Shine | 3.8 | 3.5 | 4.0 |
| Softness | 3.8 | 3.0 | 4.0 |
| Body/volume | 3.3 | 3.5 | 4.3 |
| Hair comb-through/tangling | 3.8 | 4.0 | 4.3 |
| Residue | 3.8 | 4.0 | 4.7 |
| Static | 3.5 | 4.0 | 5.0 |

For the synthetic shampoos, the subjects preferred either formulations with no carmin protein extract (BC-35-18-A—Example 8) or 0.1% carmin protein extract formulation (BC-35-18-B—Example 9) for product texture/feel, the 0.1% carmin protein extract formulation (BC-35-18-B—Example 9) was preferred with respect to static, the 0.5% carmin protein formulation (BC-35-18-C—Example 10) was preferred for overall hair feel, greasiness, shine, softness, body/volume, hair comb-through/tangling, residue and static.

### Example 17

### Consumer Assessment of Synthetic Conditioner Formulations with Different Levels of Carmin Protein Extract

4-5 study participants were asked to use the natural conditioner formulations described in examples 11-13 and then asked to rate the products on a 1 to 5 scale based on 9 separate characteristics based on the scale described in Example 14. The combined results for the synthetic conditioner formulations are provided below.

| | 0% carmin | 0.1% carmin | 0.5% carmin |
|---|---|---|---|
| | protein | protein | protein |
| | BC-35-20-A | BC-35-20-C | BC-35-20-D |
| Product texture/feel | 4.0 | 4.5 | 4.3 |
| Overall hair feel | 3.8 | 4.0 | 3.8 |
| Greasiness | 3.8 | 3.5 | 4.5 |
| Shine | 4.0 | 4.3 | 3.8 |
| Softness | 3.8 | 3.5 | 4.5 |
| Body/volume | 4.3 | 4.5 | 4.3 |
| Hair comb-through/tangling | 3.8 | 3.5 | 4.0 |
| Residue | 3.8 | 4.3 | 4.3 |
| Static | 3.8 | 3.5 | 3.8 |

For the synthetic conditioners, the subjects preferred the formulations with no carmin protein extract (BC-35-20-A—Example 11) for static, the 0.1% carmin protein extract formulation (BC-35-20-C—Example 12) was preferred with respect to product feel/texture, overall hair feel, shine, body/volume, residue, the 0.5% carmin protein extract formulation (BC-35-20-D—Example 13) was preferred for greasiness, softness, hair comb-through/tangling and static.

### Example 18

### The Level of Protection that Carmin Containing Shampoo and Conditioner Provides to Caucasian Hair Undergoing Bleaching Using Hydrogen Peroxide

A Caucasian hair fiber was shampooed for 2 minutes and rinsed with deionized water for 2 minutes. The Caucasian hair fiber was subsequently conditioned for 2 more minutes and then rinsed with deionized water for 2 more minutes. The shampooed and conditioned hair fiber was soaked in a 6% solution (v/v) of hydrogen peroxide (adjusted to pH 9) for 30 minutes and the hydrogen peroxide was removed by rinsing the Caucasian hair fiber for 2 minutes in deionized water. The hair fibers were subsequently cut into approximately 1 cm pieces and taped onto a sample holder with conductive tape. The hair sample was sputter coated with gold and palladium to ensure they were electronically conductive and images were collected at 200× and 2,000× magnification with an XL30 scanning electron microscope.

Two different shampoo and conditioner formulations from the above examples were tested. The shampoos test are S(0)—BC-35-16-A (natural shampoo without carmin protein extract —Example 2) and S(0.5)—BC-35-16-E (natural shampoo with 0.5% carmin protein extract—Example 4). The conditioners listed in the table below are C(0)—BC-35-19-A (natural conditioner without carmin protein extract—Example 5) and C(0.5)—BC-35-19-C (natural conditioner with 0.5% carmin protein extract—Example 7).

The degree of hair damage was accessed by a group of eight panelists based on the images collected using the scanning election microscope. Prior to the evaluation, all panelists were provided with a set of images of hairs with various degrees of damage and the damage score (1 to 5 with 5 being highly damaged). They are used as comparison standard for assigning the damage score to the hair samples in the present study. Average damage scores are collected in the following table.

| Shampoo | Conditioner | | |
|---|---|---|---|
| formulation | formulation | 6% H2O2 (v/v) | Damage score |
| — | — | 6% | 4.4 |
| S (0) | C (0) | 6% | 3.3 |
| S (0.5) | C (0.5) | 6% | 2.9 |

Washing the hair sample with the 0.5% carmin natural shampoo (BC-35-16-E—Example 4) and 0.5% carmin natural conditioner (BC-35-19-C—Example 6) prior to bleaching had the highest level of protection against hair damage when the Caucasian hair was treated with a 6% (v/v) hydrogen peroxide solution.

### Example 19

### The Level of Protection that Carmin Containing Shampoo and Conditioner Provides to Caucasian Hair Undergoing Bleaching Using a Commercial Bleaching Product

A Caucasian hair fiber was shampooed for 2 minutes and rinsed with deionized water for 2 minutes. The Caucasian hair fiber was subsequently conditioned for 2 more minutes and then rinsed with deionized water for 2 more minutes. The shampooed and conditioned hair fiber was soaked in commercial developer crème from Gamier® Belle Color™ 80 Medium Blonde for 30 minutes and the developer crème was removed by rinsing the Caucasian hair fiber for 2 minutes in deionized water. The hair fibers were subsequently cut into approximately 1 cm pieces and taped onto a sample holder with conductive tape. The hair sample was sputter coated with gold and palladium to ensure they were electronically conductive and images were collected at 200× and 2,000× magnification with an XL30 scanning electron microscope.

Two different shampoo and conditioner formulations from the above examples were tested. The shampoos tests are S(0)—BC-35-16-A (natural shampoo without carmin protein extract —Example 2) and S(0.5)—BC-35-16-E (natural shampoo with 0.5% carmin protein extract—Example 4). The conditioners listed in the table below are C(0)—BC-35-19-A (natural conditioner without carmin protein extract—Example 5) and C(0.5)—BC-35-19-C (natural conditioner with 0.5% carmin protein extract—Example 7).

The degree of hair damage was accessed by a group of eight panelists based on the images collected using the scanning electron microscope. Prior to the evaluation, all panelists were provided with a set of images of hairs with various degrees of damage and the damage score (1 to 5 with 5 being highly damaged). They are used as comparison standard for assigning the damage score to the hair samples in the present study. Average damage scores are collected in the following table.

| Shampoo | Conditioner | Developer Crème in | |
|---|---|---|---|
| formulation | formulation | Garnier ® Belle Color ™ | Damage score |
| S (0) | C (0) | 80 Medium Blonde | 4.1 |
| S (0.5) | C (0.5) | 80 Medium Blonde | 2.3 |

Washing the hair sample with the 0.5% carmin protein extract natural shampoo (BC-35-16-E—Example 4) and 0.5% carmin protein extract natural conditioner (BC-35-19-C—Example 6) prior to bleaching had the highest level of protection against hair damage when the Caucasian hair was treated with a commercial developer crème.

### Example 20

### The Level of Protection that Carmin Containing Shampoo and Conditioner Provides to Asian Hair Undergoing Bleaching Using Hydrogen Peroxide

An Asian hair fiber was shampooed for 2 minutes and rinsed with deionized water for 2 minutes. The Asian hair fiber was subsequently conditioned for 2 more minutes and then rinsed with deionized water for 2 more minutes. The shampooed and conditioned hair fiber was soaked in a 6% solution (v/v) of hydrogen peroxide (adjusted to pH 9) for 30 minutes and the hydrogen peroxide was removed by rinsing the Asian hair fiber for 2 minutes in deionized water. The hair fibers were subsequently cut into approximately 1 cm pieces and taped onto a sample holder with conductive tape. The hair sample was sputter coated with gold and palladium to ensure they were electronically conductive and images were collected at 200× and 2,000× magnification with an XL30 scanning electron microscope.

Two different shampoo and conditioner formulations from the above examples were tested. The shampoos test are S(0)—BC-35-16-A (natural shampoo without carmin protein extract—Example 2) and S(0.5)—BC-35-16-E (natural shampoo with 0.5% carmin protein extract—Example 4). The conditioners listed in the table below are C(0)—BC-35-19-A (natural conditioner without carmin protein extract—Example 5) and C(0.5)—BC-35-19-C (natural conditioner with 0.5% carmin protein extract—Example 7). The degree of hair damage was accessed by a group of eight panelists based on the images collected using the scanning election microscope. Prior to the evaluation, all panelists were provided with a set of images of hairs with various degrees of damage and the damage score (1 to 5 with 5 being highly damaged). They are used as comparison standard for assigning the damage score to the hair samples in the present study. Average damage scores are collected in the following table.

| Shampoo | Conditioner | | |
|---|---|---|---|
| formulation | formulation | 6% H₂O₂ (v/v) | Damage score |
| — | — | — | 1.6 |
| — | — | 6% | 3.6 |
| S (0) | C (0) | 6% | 3.9 |
| S (0.5) | C (0.5) | 6% | 3.1 |

Washing the hair sample with the 0.5% carmin protein extract natural shampoo (BC-35-16-E—Example 4) and 0.5% carmin protein extract natural conditioner (BC-35-19-C) prior to bleaching had the highest level of protection against hair damage when the Asian hair was treated with a 6% (v/v) hydrogen peroxide solution.

### Example 21

### The Level of Protection that Carmin Containing Shampoo and Conditioner Provides to Asian Hair Undergoing Bleaching Using a Commercial Bleaching Product

An Asian hair fiber was shampooed for 2 minutes and rinsed with deionized water for 2 minutes. The Asian hair fiber was subsequently conditioned for 2 more minutes and then rinsed with deionized water for 2 more minutes. The shampooed and conditioned hair fiber was soaked in commercial developer crème from Gamier® Belle Color™ 80 Medium Blonde for 30 minutes and the developer crème was removed by rinsing the Asian hair fiber for 2 minutes in deionized water. The hair fibers were subsequently cut into approximately 1 cm pieces and taped onto a sample holder with conductive tape. The hair sample was sputter coated with gold and palladium to ensure they were electronically conductive and images were collected at 200× and 2,000× magnification with an XL30 scanning electron microscope.

Two different shampoo and conditioner formulations from the above examples were tested. The shampoos test are S(0)—BC-35-16-A (natural shampoo without carmin protein extract—Example 2) and S(0.5)—BC-35-16-E (natural shampoo with 0.5% carmin protein extract—Example 4). The conditioners listed in the table below are C(0)—BC-35-19-A (natural conditioner without carmin protein extract—Example 5) and C(0.5)—BC-35-19-C (natural conditioner with 0.5% carmin protein extract—Example 7).

The degree of hair damage was accessed by a group of eight panelists based on the images collected using the scanning electron microscope. Prior to the evaluation, all panelists were provided with a set of images of hairs with various degrees of damage and the damage score (1 to 5 with 5 being highly damaged). They are used as comparison standard for assigning the damage score to the hair samples in the present study. Average damage scores are collected in the following table.

| Shampoo | Conditioner | Developer Crème in | |
|---|---|---|---|
| formulation | formulation | Garnier ® Belle Color ™ | Damage score |
| — | — | — | 1.6 |
| — | — | 80 Medium Blonde | 2.6 |
| S (0) | C (0) | 80 Medium Blonde | 2.9 |
| S (0.5) | C (0.5) | 80 Medium Blonde | 1.9 |

Washing the hair sample with the 0.5% carmin protein extract natural shampoo (BC-35-16-E—Example 4) and 0.5% carmin protein extract natural conditioner (BC-35-19-C—Example 7) prior to bleaching had the highest level of protection against hair damage when Asian hair was treated with a commercial developer crème.

### Example 22

### Preparation of a Natural Cream Base Suitable to Incorporate Carmin Protein Extract (BC-37-3-A)

| Ingredients | % |
|---|---|
| Phase A | |
| Water | 73.2 |
| Phase B | |
| Keltrol ® CG-SFT (Xanthan Gum) | 0.4 |
| Genuvisco ® CG-131 (Carrageenan) | 0.8 |
| Glycerin | 3.0 |
| Phase C | |
| Lipocol ® C (Cetyl Alcohol) | 7.0 |
| Shea Butter | 1.0 |
| Cetiol ® OE | 3.0 |
| Olive Oil | 3.0 |
| Tocopherol | 0.1 |
| Phase D | |
| Hydresia ® SF-2 | 5.0 |
| Phase E | |
| Glycerin | 2.0 |
| Phase F | |
| Preservation | 1.5 |

Mix ingredients of phase B until homogenous and add to Phase A under strong agitation and mix 30 minutes. Heat to 55-60° C. Weight together ingredients from Phase C and heat to 55-60° C. while mixing. Add Phase C to A+B and homogenize at 5,000 rpm for 10 minutes while maintaining the temperature. Lower the homogenization speed to 3,000 rpm and add phase D. Mix 10 minutes. Start cooling using side scraper for mixing. At 45° C., add phase E that has already been pre-mixed until homogenous. Mix 10 minutes. Add phase F and mix 10 minutes. Adjust the pH between 5.5-6.0 and continue cooling. The cream base is suitable to include 0.01% to 10.0% (w/w) carmin protein extract, for example, by using glycerin (Phase E) as a diluent.

### Example 23

### Preparation of a Synthetic Cream Base Suitable to Incorporate Carmin Protein Extract (BC-37-3-A)

| Ingredients | % |
|---|---|
| Phase A | |
| Water | 86.8 |
| Disodium EDTA | 0.1 |
| Phase B | |
| Carbopol ® Ultrez-21 | 0.3 |
| Phase C | |
| Lipomulse ® 165 | 3.0 |
| Lipocol ® C (Cetyl Alcohol) | 0.8 |
| Stearic Acid | 2.0 |
| Liponate ® GC | 1.5 |
| Cetiol ® CC | 1.5 |
| Tocopherol | 0.1 |
| Phase D | |
| Glycerin | 2.0 |
| Phase E | |
| Preservation | 1.0 |
| Phase F | |
| Triethanolamine | 0.15 |

Add ingredients of phase B to Phase A under without agitation and let stand 5 minutes. Start agitation with a propeller and heat to 75° C. Weight together ingredients from Phase C and heat to 75° C. while mixing. Add Phase C to A+B mixture and homogenize at 5,000 rpm for 10 minutes while maintaining the temperature. Start cooling using side scraper for mixing. At 45° C., add phase D that has already been pre-mixed until homogenous. Mix 10 minutes. Add phase E and mix 10 minutes. Adjust the pH between 5.5-6.0 with phase F and continue cooling. The cream base is suitable to include 0.01% to 10.0% (w/w) carmin protein extract, for example, by using glycerin (Phase D) as a diluent.

### Example 24

### Amino Acid Composition of Carmin Protein Extract

A carmin protein extract was prepared from Carthamus tinctorius seed using an aqueous protein extraction process and the amino acid composition of the extract was analyzed. The results are presented in the following table.

| | Approximate Amino Acid Percentage |
|---|---|
| Amino Acid | (as a percentage of total) |
| Aspartic Acid | 10.08 |
| Glutamic Acid | 22.05 |
| Serine | 4.50 |
| Glycine | 5.38 |
| Histidine | 3.50 |
| Arginine | 11.45 |
| Threonine | 3.22 |
| Alanine | 4.23 |
| Proline | 4.27 |
| Tyrosine | 3.37 |
| Valine | 4.66 |
| Methionine | 1.75 |
| Cystine | 1.42 |
| Isoleucine | 3.94 |
| Leucine | 6.66 |
| Phenylalanine | 4.94 |
| Lysine | 3.26 |
| Tryptophan | 1.32 |

### Example 25

### Preparation of a Natural Shampoo with 0.5% Carmin Protein Extract (BC-35-24)

| Ingredients | % |
|---|---|
| Phase A | |
| Water | 52.0 |
| Phase B | |
| Glycerin | 5.0 |
| Chrondus Crispus (Carrageenan) (Genuvisco ® | 0.4 |
| CG-131, CP Kelco) | |
| Xanthum Gum (Keltrol ® CG SFT, CP Kelco) | 0.8 |
| Carmin Protein Extract | 0.5 |
| Phase C | |
| Decyl Glucoside (Plantaren ® 2000, BASF) | 20.0 |
| Lauryl Glucoside (Plantaren ® 1200, BASF) | 10.0 |
| Hydrogenated Jojoba Esters (Floraester K-20W, | 1.25 |
| Floratech) | |
| Phase D | |
| Carthamus tinctorius (safflower) oleosomes + | 5.0 |
| water (Hydresia ® SF2 | |
| Passiflora Edulis Seed Oil, Ortza Sativa Rice Bran | 1.5 |
| Oil, Euterpe Oleracea Fruit Oil (Bearcare ARS | |
| Hair System, Bereca) | |
| Fragrance | 0.3 |
| Phase E | |
| Gluconolactone + Sodium Benzoate (Geogard ® | 1.5 |
| Ultra, Lonza) | |
| Phase F | |
| Citric Acid | 1.75 |

Premix ingredients of Phase B and add to Phase A under strong agitation and mix for 30 minutes. Add ingredients from Phase C in order of appearance. Mix until homogenous. Heating to 40-45° C. will accelerate the process. Mix together the ingredients from Phase D at 400 rpm for 20 minutes and add to batch. Adjust the pH between 3.8-4.2 using Phase F after 24 hours. Viscosity of the batch is 5,000 cps using the Brookfield RVP Spindle #5 at 20 rpm.

### Example 26

### Preparation of a Natural Conditioner with 0.5% Carmin Protein Extract (BC-35-25)

| Ingredients | % |
|---|---|
| Phase A | |
| Water | 76.95 |
| Arginine | 0.25 |
| Panthenol, (D-Panthenol 75W, BASF) | 0.5 |
| Phase B | |
| Brassicyl Isoleucinate Esylate Brassica | 8.0 |
| Alcohol (Emulsence ™ HC, Inolex) | |
| Butyrospermum Parkii (Shea Butter) (Shea | 2.0 |
| Butter Ultra Refined, Bio Botanica) | |
| Passiflora Edulis Seed Oil, Oriza Sativa Rice | 2.0 |
| Bran Oil, Euterpe Oleracea Fruit Oil | |
| (Beracare ARS Hair System, Beraca) | |
| Phase C | |
| Carthamus tinctorius (safflower) oleosomes + | 5.0 |
| water (Hydresia ® SF2) | |
| Fragrance | 0.3 |
| Phase D | |
| Geogar ® Ultra | 1.5 |
| Phase E | |
| Glycerine | 3.0 |
| Carmin Protein Extract | 0.5 |

Add all ingredients of Phase A to the water with mixing. Heat to 75° C. In a separate container, weigh all ingredients of Phase B together and heat to 75° C. Add Phase B to Phase A under strong agitation. Mix 20 minutes at 75° C. then slow agitation and start cooling. Mix together the ingredients from Phase C at 400 rpm for 20 minutes and add to the batch when the temperature is under 45° C. Add ingredients from Phase D and mix well. Continue to cool. Mix together ingredients of Phase E and add to the batch. Mix for 10 minutes. Adjust the pH between 3.8-4.2 after 24 hours. Final viscosity of the batch is 15,000 cps using the Brookfield RVP Spindle #6 at 20 rpm.

### Example 27

### Preparation of a Natural Shower Gel with 0.5% Carmin Protein Extract (BC-35-23)

| Ingredients | % |
|---|---|
| Phase A | |
| Water | 48.5 |
| Phase B | |
| Glycerine | 5.0 |
| Chrondus Crispus (Carrageenan) (Genuvisco ® | 0.4 |
| CG-131, CP Kelco) | |
| Xanthan Gum (Keltrol ® CG SFT, CP Kelco) | 0.8 |
| Carmin Protein Extract | 0.5 |
| Phase C | |
| Decyl Glucoside (Plantaren ® 2000, BASF) | 20.0 |
| Lauryl Glucoside (Plantaren ® 1200, BASF) | 10.0 |
| Hydrogenated Jojoba Esters (Floraester ® K- | 1.25 |
| 20W, Floratech) | |
| Phase D | |
| Carthamus tinctorius (safflower) oleosomes + | 5.0 |
| water (Hydresia ® SF2) | |
| Fragrance | 0.3 |
| Phase E | |
| Glyconactone + Sodium Benzoate (Geogard ® | 1.5 |
| Ultra, Lonza) | |
| Cocamidopropyl Betaine (Dehyton ® pK 45, | 5.0 |
| BASF) | |
| Phase F | |
| Citric Acid 25% | 1.75 |

Premix ingredients of Phase B and add to Phase A under strong agitation and mix for 30 minutes. Add ingredients from Phase C in order of appearance. Mix until homogenous. Heating to 40-45° C. will accelerate the process. Mix together the ingredients from Phase D at 400 rpm for 20 minutes and add to the batch. Add ingredients from Phase E in the order of appearance and mix well between each addition. Once all ingredients from Phase E are added mix at 600-800 rpm for 20 minutes. Adjust the pH between 3.8-4.2 with Phase F after 24 hours. Final viscosity of the batch is 6,000 cps using the Brookfield RVP Spindle #5 at 20 rpm.

### Example 28

### Comparative Study for the Hydration Potential of Body Washes Over a Period of 8 Days

The primary aim of the study was to evaluate the cutaneous hydration of two Body Wash Products (Product A—Dove® Deep Moisture Nourishing Body Wash and Product B—Body wash with 0.5% carmin protein extract (BC-35-25 as described above in Example 27). The test products were evaluated for their effect on skin hydration over a period of 7 days.

A total of 20 healthy female volunteers were recruited for the study. Volunteers were not allowed to apply any topical products (e.g., body wash, creams, lotion) on their legs starting 7 days prior to the beginning of the study. On Day 0, volunteers arrived at the laboratory at least 2 hours after taking their shower or bath. After at least 30 minutes in the laboratory room (for skin acclimatization), hydration measurements were taken from 3 different test sites of 2×3 cm on each calf for each volunteers (T=0). Hydration measurements were taken with a Corneometer® CM825 (Courage & Khazaka, Germany) equipped with a 49 mm² probe. The probe was gently pressed against the skin (a pressure of 3.56 N) and the capacitance was recorded. Five consecutive measurements were taken from each test site at each time measurement and the average was noted.

After baseline measurements (time T=0), the test products were applied onto the appropriate calf which were distributed at random in order to reduce systemic errors related to anatomical differences between volunteers. The amount of product applied was 2 mg/cm² and the test products were rinsed off just after application. After 30 minutes, the same hydration measurements were taken (T=30 min). Then, then 2 body wash products were given to volunteers. During the week of the study, the volunteers had to apply each test product once a day according to the same randomization as the first measurements (one test body wash on one leg). Also, the use of any topical products (e.g., creams, lotions, soap, or a body wash other than the 2 test products) was strictly prohibited on the test areas. Volunteers were asked to take their last shower or bath with the 2 products on the day D=6 in the evening. The last measurements (T=7 days) were taken from each test site, after a skin acclimatization period of at least 30 minutes in the laboratory room. The average hydration results for the 2 products (expressed in arbitrary units) are presented in the table below.

| Hydration average at each | | | |
|---|---|---|---|
| time of measurement | T = 0 | T = 30 minutes | T = 7 days |
| Product A | 23.2 | 30.2 | 23.3 |
| Product B | 24.0 | 30.0 | 25.5 |

At T=0, before applying the test products, no significant difference was observed between the 2 calves. The test Product A (Dove® Deep Moisture Nourishing Body Wash) and Product B (Body wash with 0.5% carmin protein extract (BC-35-25 as described above in Example 27) significantly increased skin hydration 30 minutes after application (p<0.01), when compared to the mean value of results obtained from initial readings at T=0. However, both products did not show a significant difference at Day 7, when compared to the mean value of the results obtained from initial readings at T=0. Additionally, statistical analysis demonstrated no difference between the two test products at T=30 minutes and at T=7 days.

1. A personal care formulation comprising a carmin protein and a diluent, carrier or excipient suitable for use in a personal care formulation.

2. The personal care formulation according to claim 1 wherein the carmin protein is in an extract obtainable from Carthamus tinctorius.

3. The personal care formulation according to claim 2 wherein the carmin protein extract comprises less than about 40% (w/w) of other seed proteins.

4. The personal care formulation according to claim 1, wherein the formulation comprises at least about 0.1% (w/w) of the carmin protein or carmin protein extract.

5. The personal care formulation according to claim 1, wherein the formulation comprises from about 0.1% (w/w) to about 10% (w/w) of the carmin protein or carmin protein extract.

6. The personal care formulation according to claim 2, wherein the carmin protein extract is an extract comprising from about 5% to about 50% endogenous Carthamus plant oils.

7. The personal care formulation according to claim 1, wherein the excipient is selected from the group consisting of emollients, moisturizers, pH-modifying agents, viscosity modifying agents, surfactants and active agents.

8. The personal care formulation according to claim 1, wherein the personal care formulation is selected from the group consisting of a skin care formulation, a bath and body formulation, a sun care formulation, a make-up formulation a shaving formulation or a hair care formulation, or a hair care formulation.

9.-11. (canceled)

12. The personal care formulation according to claim 8, wherein the hair care formulation is selected from the group consisting of a shampoo, a conditioner, a re-conditioner, a mousse, a gel, a hair spray, a pomade, a hair waving agent, a hair dye, a hair coloring formulation, a hair lightening formulation and a hair bleaching formulation.

13.-15. (canceled)

16. A method of formulating a personal care formulation comprising:
(a) providing a carmin protein;
(b) providing a diluent, carrier or excipient; and
(c) contacting the carmin protein with the diluent, carrier or excipient to formulate a personal care formulation.

17. The method according to claim 16 wherein the carmin protein is in an extract obtainable from Carthamus tinctorius.

18. The method according to claim 17 wherein the carmin protein extract comprises less than about 40% (w/w) of other seed protein.

19. The method according to claim 16, wherein the formulation comprises at least about 0.1% (w/w) of the carmin protein or carmin protein extract.

20. The method according to claim 16, wherein the formulation comprises from about 0.1% (w/w) to about 10% (w/w) of the carmin protein or carmin protein extract.

21. The method according to claim 17, wherein the carmin protein extract is an extract comprising from about 5% to about 50% endogenous Carthamus plant oils.

22. The method according to claim 16, wherein the excipient is selected from the group consisting of emollients, moisturizers, pH-modifying agents, viscosity modifying agents, surfactants and active agents.

23. The method according to claim 16, wherein the formulation is selected from the group consisting of a skin care formulation, a bath and body formulation, a sun care formulation, a make-up formulation, a shaving formulation, or a hair care formulation.

24.-26. (canceled)

27. The method according to claim 17, wherein the hair care formulation is selected from the group consisting of a shampoo, a conditioner, a re-conditioner, a mousse, a gel, a hair spray, a pomade, a hair waving agent, a hair dye, a hair coloring formulation, a hair lightening formulation and a hair bleaching formulation.

28. The method according to claim 17, wherein the hair care formulation is product capable of preventing or repairing damaged hair.

29.-40. (canceled)
